(19) United States
(12) Patent Application Publication
(10) Pub. No.: US 2005/0019939 A1
(43) Pub. Date: Jan. 27, 2005

(54) Combination marker for liquids and method identification thereof

(76) Inventors: Dale Spall, Los Alamos, NM (US); Todd M. Allen, Los Alamos, NM (US); Roy Goeller, Los Alamos, NM (US); Peter Kottenstette, Dallas, TX (US)

Correspondence Address: George R. Schultz;Schultz & Associates, P.C., One Lincoln Centre, 5400 LBJ Freeway, Suite 525, Dallas, TX, 75240, US

(21) Appl. No.: 10/628,072

(22) Filed: Jul. 25, 2003

(51) Int. Cl. G01N 33/00

(52) U.S. Cl. 436/139

(57) ABSTRACT

A formulation of markers for the identification of liquids is provided. Formulation includes a marker which has a high molar absorptivity in the wavelength range of 600-1000 nm. The invention further provides for a combination marker including a marker with known absorbtivity within a wavelength range and a molecular marker including various molecular formulations and isotopic markers used in conjunction. The invention further provides a method of testing for a marked liquid which employs the testing for an absorbance marker as a screening mechanism to reduce the number of tests for the molecular marker required to assure the unadulterated nature of the liquid.

### FIELD OF THE INVENTION

The present invention is related to liquid identification. In particular, the present invention involves methods for marking a liquid and methods and systems for the identification of a liquid using a combination of two types of markers, namely a marker that has a relatively high absorptivity in the wavelength range of 600-1000 nm and a molecular marker.

### BACKGROUND OF THE INVENTION

There are many reasons to mark liquids for identification. One reason is the identification of petroleum products. Often markers are used to differentiate among various grades of petroleum products. For example, markers are used to distinguish premium from regular grade gasoline, or aviation gasoline from motor gasoline. Markers are also widely employed to identify proprietary or specific brands of petroleum product from a particular refiner. Further, markers are sometimes required by government regulation to monitor the tax classification of diesel fuel, home heating oil, and kerosene's; in the case of tax classification monitoring, markers are used for the identification of petroleum products that might be substituted illegally for more costly or higher taxed fuels. Other reasons for identifying particular fuels are determination of theft, or locating leaks from storage facilities at service stations, oil terminals, large scale industrial lubrication systems, or automotive transmission and hydraulic systems.

Most petroleum product markers have several important chemical properties. They are miscible in petroleum product in which the marker is being used, difficult to remove from the product by any simple treatment (e.g. filtering, bleaching, reactive conversion), easily identifiable and, in all cases, identifiable by the user.

Prior art markers are often organic dyes, that is, materials with strong absorbency in the visible portion of the electromagnetic spectrum. Thus, a fuel containing such organic dyes is immediately identifiable visually. Red-26, is an example of such a marker. Red-26 is used by the Internal Revenue Service in the United States to distinguish nontaxed home heating oil from other taxed fuels of similar composition. If red dye is present, no federal taxes are paid on the product. Therefore, presence of the red dye in the product is taken as evidence of tax evasion if the fuel is sold as taxed commercial diesel.

In many cases, however, it is desirable that the marker not be visible in the liquid. Markers that are not visibly discernible are known in the industry as “silent markers.” Silent markers identify a product but do not provide any visual indication of the identity of the product.

In order to detect the presence and concentration of many of the prior art silent markers, the markers must be extracted from the liquid by a chemical process. For example, the addition of a chemical reagent to the liquid causes the marker to turn to a visibly distinct color. The depth of the color indicates the concentration of the marker present in the liquid. A laboratory measurement with a spectrometer can provide a more precise measure of the concentration of the marker if needed. Such chemical manipulation is time consuming and expensive and requires trained personnel to execute.

In contrast, some silent markers are large organic molecules that either absorb light of a specific frequency or fluoresce when exposed to certain wavelengths of light. The fluorescence or absorbance is used to indicate the presence of the marker and its concentration in the liquid. The presence of such a marker is detected by exposing the marker to a chosen frequency of light and then detecting the fluorescent light or the absorbed light with a detection system. Markers which rely on absorbance and fluorescence both suffer from the limitation of being unreliable when exact identification of concentration is required. Also these markers can be changed and counterfeited relatively easily thus adding to their unreliability.

U.S. Pat. No. 5,958,780 to Asher et al, entitled Method For Marking And Identifying Liquids, discloses the use of a ratio between the absorbance of two separate markers to identify liquids. Each of the markers disclosed demonstrates an absorbance spectrum with at least one wavelength range differentiable from the absorbance of the background liquid. However, Asher requires using at least two markers, measuring the absorbance of the two markers to create a ratio, and then comparing that ratio to a look table of predetermined values to provide information concerning the identity of the liquid. Also, Asher does not disclose or suggest a marker with a high molar absorptivity.

Molecular marking is a precise method of determining whether or not a liquid has been adulterated or altered. However, testing molecular markers requires all the suspected liquids be analyzed in a laboratory which is costly and time consuming.

U.S. Pat. No. 5,981,283 to Anderson II et al, entitled Method Of Tagging Hydrocarbon Fuels, discloses the use of a tagging agent that is elutable by chromatographic analysis of a liquid hydrocarbon fuel composition apart from the hydrocarbon components of the fuel composition. The tagging agent is incorporated in the fuel composition in a known concentration. Later analysis of the concentration of the tagging agent in the fuel composition indicates whether or not the fuel composition has been diluted. Because a gas chromatograph is required for the analysis of the tagging agent, Anderson II et al, requires the analysis is performed in a laboratory and not on site in the field. U.S. Pat. No. 5,981,283 is hereby incorporated by reference.

U.S. Pat. No. 5,474,937 to Anderson II et al, entitled Method Of Identifying Chemicals By Use Of Non-Radioactive Isotopes, discloses the use of a tagging agent comprised of a chemical element or an organic compound with one or more atoms that are non-radioactive isotopes found in nature at extremely low levels. A small quantity of the isotopic compound is introduced into the storage vessel containing the chemical to be transported prior to shipment of the chemical. Upon arrival at its destination point, a sample of the chemical shipment is analyzed. Matching the isotopic compound found in the chemical with the isotopic compound introduced into the storage vessel prior to shipment is indicative that the shipped chemical is identical to the chemical received. Non-radioactive materials may further be employed for detecting the source of a newly introduced contaminant in a water supply. Because a gas chromatograph is required for the analysis of the tagging agent, Anderson II et al, requires the analysis is performed in a laboratory and not on site in the field. U.S. Pat. No. 5,474,937 is hereby incorporated by reference.

What is needed is a method of marking, tagging, or otherwise identifying adulterated or altered liquids which allows for the quick and qualitative identification of the concentration of an added marker to reduce the number of lab test required to analyze a large set of samples of suspected liquids. Also, it would be beneficial if the markers were relatively inexpensive and available in a wide variety of forms to provide a wide variety of markers.

### SUMMARY OF THE INVENTION

The present invention provides a method for marking and identifying liquids by using an absorption marker that possesses a large molar absorptivity in the wavelength range of about 600-1000 nm and a combination marker comprised of a lower reliability absorption marker and a higher reliability molecular marker.

In the preferred embodiment of the invention the absorption marker includes metal containing and metal free phthalocyanine markers, metal containing and metal free naphthalocyanine markers, squarilium markers, croconic acid markers, indole and substituted indole cyanine and carbocyanine markers, thiazole type cyanine and carbocyanine markers, oxazole type cyanine and carbocyanine markers, metal dithiolene complexes, and indoaniline metal complexes.

In the preferred embodiment the molecular marker includes two classes of suitable materials which can be used as markers. The first is polynuclear aromatic hydrocarbons and halogenated hydrocarbons. Examples of suitable markers include 1,2-diphenylbenzene; 1,4-diphenylbenzene, triphenylmethane, 1,3,5-triphenylbenzene, 1,1,2-triphenylethylene; tetraphenylethylene, 1,2,3,4-tetrahydrocarbazole, 1,3-diphcnylacetone, 2-chlorobenzophenone; 4,4′-dichlorobenzophenone; 4-benzoylphenone; 4-bromobenzophenone; 4-methoxybenzophenone; 4-methylbenzophenone; 9-fluorenone; 1-phenylnaphthalene; 3,3′dimethoxybiphenyl; and 9-phenylanthracene. The second is a group of isotopic markers whose molecular weight has been artificially adjusted to be detectable.

Both the absorption markers and the molecular markers may be added in small concentrations and do not affect the physical or chemical properties of the liquids to which they are added.

In practice the disclosed method provides a process to identify a fluid efficiently and exactly which is lacking in the prior art. The low reliability absorption marker allows for an efficient initial screening of the liquid being tested to determine if the marker is inside a predetermined tolerance. The high reliability molecular marker allows for an exact determination of the concentration of marker present thus allowing an exact identification of the liquid.

The present invention is an improvement over the prior art because due to the absorption marker having a high molecular absorbability, the absorption marker can be detected in low concentrations in the liquid. The absorption marker is easily identifiable by relatively quick and simple means using a simple IR spectrometer, and reduces the overall number of samples sent to the laboratory to determine if the liquid has been adulterated or altered. The method is more efficient and cost effective that the prior art.

The invention will be better understood from the following more detailed description taken in conjunction with the accompanying drawings.

### DETAILED DESCRIPTION OF THE DRAWINGS

A better understanding of the invention can be obtained from the following detailed description of exemplary embodiments as considered in conjunction with the following drawings in which:

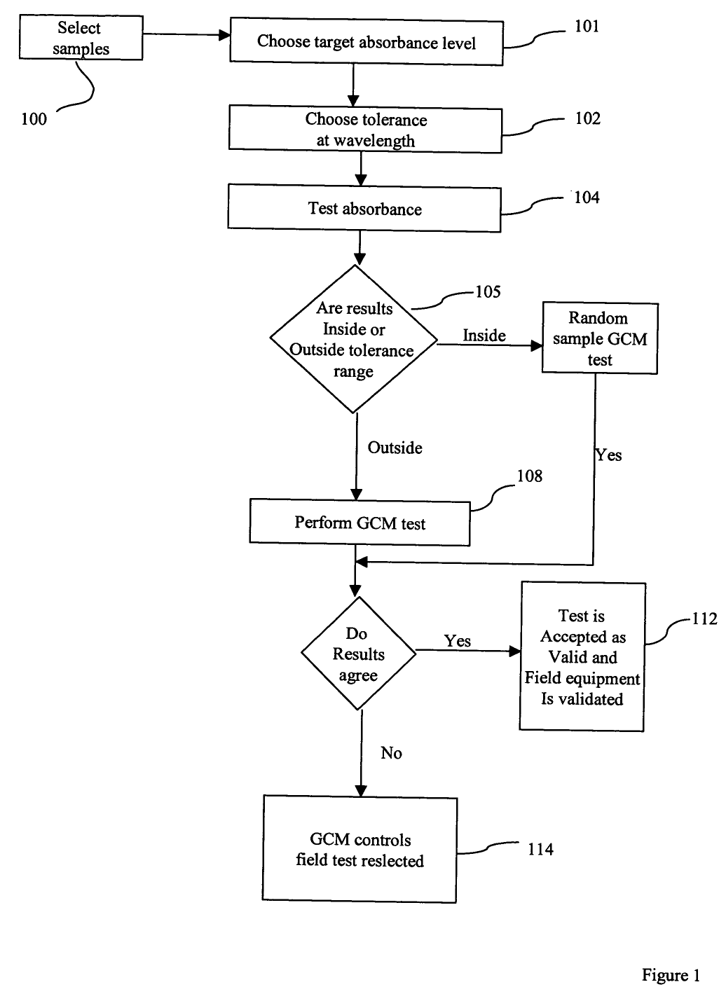

FIG. 1 depicts the steps of testing a liquid for markers; and

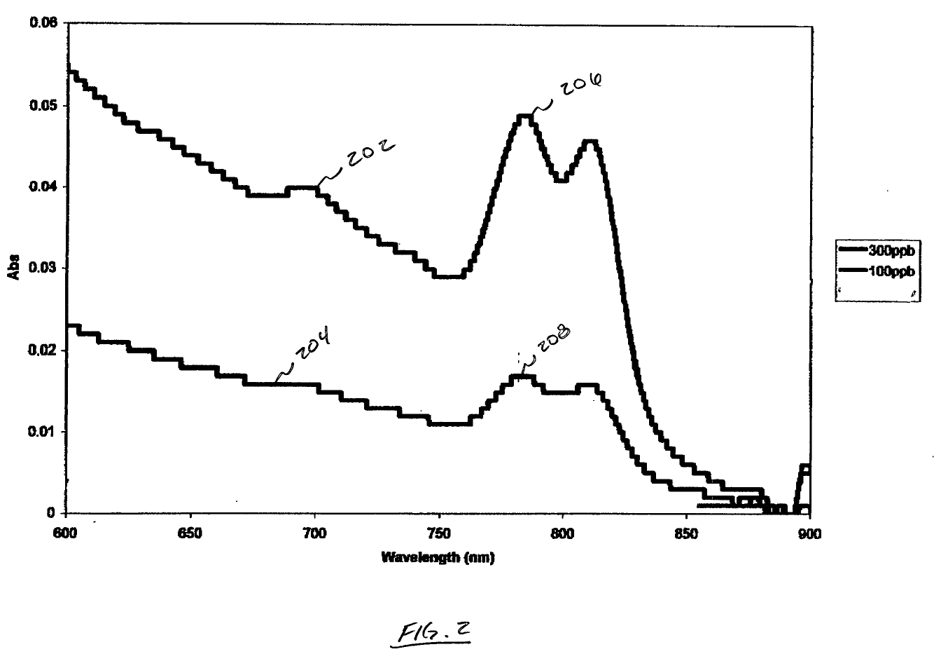

FIG. 2 is a plot of the absorbance of two samples of a naphthalocyanine.

### DETAILED DESCRIPTION OF EXEMPLARY EMBODIMENTS

The present invention provides a method for identifying liquids that have been adulterated or altered by marking the liquid with lower reliability absorption marker and a higher reliability molecular marker.

Absorption Marker

The absorption marker possesses a molecular absorptivity of approximately 5 times 10⁴ L mole⁻¹ cm⁻¹ and preferably greater than approximately 10⁵ L mole⁻¹ cm⁻¹ in the wavelength range of 600-1000 nm. The marker can be detected in low concentrations of the liquid using a commercially available IR spectrometer. Also, the marker should produce an easily recognizable characteristic peak at a known wavelength.

Many marker classes have the above required characteristics and are relatively inexpensive. By way of illustration and not as a limitation, the following absorption marker classes are suitable for use in the invention: metal containing and metal free phthalocyanine markers, metal containing and metal free naphthalocyanine markers, squarilium markers, croconic acid markers, indole and substituted indole cyanine and carbocyanine markers, thiazole type cyanine and carbocyanine markers, oxazole type ∞cyanine and carbocyanine markers, metal dithiolene complexes, and indoaniline metal complexes.

Because many of the absorption markers are solids, before the markers are added to the liquid to be marked, the absorption marker is mixed with a solvent. The solvent should be non-reactive and totally miscible with the liquid. If a solvent is to be used, it is preferable the solvent is the liquid itself. For example, to mix a carbocyanine marker with diesel fuel, the diesel fuel would be the solvent.

The concentration of absorption marker in the solvent is based on the desired concentration of the absorption marker in the liquid. Preferably, the marker is added in such an amount such that the concentration of the absorption marker in the liquid is 1 ppb-10 ppm and thereby produces a readily identifiable peak. According to Beer's Law, the absorbance due to the absorption marker is directly related to the concentration of the absorption marker in the liquid. Beer's law states:
A=εbC,
where A is the integrated absorbance, ε is the molar absorptivity, b is the path length in centimeters, and C is the molar concentration. The higher the molar absorptivity, the lower the concentration need to be to obtain a desired integrated absorbance with low concentrations comes a savings in cost.

The upper range of the concentration is almost unlimited but is preferred to be 10 ppm or less due to cost of the marker. In the preferred embodiment the concentration range is 20 ppb-1 ppm and depends on the molar absorptivity of the selected marker and the commercial cost of the marker. Notably, the marker does not settle out or separate and therefore, the concentration remains reasonably constant.

The following specific, non-limiting examples illustrate the methods used in preparing the compounds which are useful in the practice of the invention.

### EXAMPLE 1

A 0.2541 gram sample of a metal free phthalocyanine having a molecular weight of 966 was dissolved in 250 ml of diesel to produce a marker fuel solution. The marker fuel solution was serially diluted four times by a factor of 10 by volumetrically extracting a 25 ml sample of the marker fuel solution, and then inserting the extracted marker fuel solution into a volumetric flask. Next, diesel fuel is added to the volumetric flask to bringing the volume in the volumetric flask up to 250 ml. The final concentration of the metal free phthalocyanine was 102 ppb weight to volume. An absorption spectra of the sample was run on a Shimadzu model UV-3101PC spectrometer measuring from 600 to 1000 nm in the double beam mode with untreated diesel as the reference. The absorption maximum was 712 nm, and the absorbance was 0.166, with a background absorbance of 0.005. The calculated molar absorptivity for the compound is 156000.

### EXAMPLE 2

A 0.2817 gram sample of a metal free naphthalocyanine with a molecular weight of 852 was dissolved in 250 ml of kerosene to produce a marker fuel solution. The marker fuel solution was serially diluted four times by a factor of 10 by volumetrically extracting a 25 ml sample of the marker fuel solution, and then inserting the extracted marker fuel solution into a volumetric flask and then adding kerosene to the volumetric flask to bringing the volume in the volumetric flask up to 250 ml. The final concentration of the metal free naphthalocyanine in the kerosene was 132 ppb weight to volume. An absorption spectra of the sample was run on a Shimadzu model UV-3101PC spectrometer measuring from 600 to 1000 nm in the double beam mode with untreated kerosene as the reference. The absorption maximum was 768 nm and the absorbance was 0.214. The sample was diluted by a factor of two, yielding an absorbance of 0.105. Further dilutions by a factor of two yielded absorbance readings of 0.050 and 0.022. The final concentration was 16 ppb, and the signal to noise ratio at the max wavelength was 4.6. The concentration curve varied linearly with the concentration, showing agreement with Beers Law.

### EXAMPLE 3

A sample weighing 0.3625 grams of a metal free, substituted dimethylindole cyanine was dissolved in 250 ml of toluene to produce a marker toluene solution. The marker toluene solution was serially diluted four times by a factor of 10 by volumetrically extracting a 25 ml sample of the marker toluene solution, and then inserting the extracted marker tolerance solution into a volumetric flask. Next, gasoline was added to the volumetric flask to bringing the volume in the volumetric flask up to 250 ml, to yield a final concentration of the metal free, substituted dimethylindole cyanine in the gasoline at 305 ppb. An absorption spectra of the sample was run on a Shimadzu model UV-3101PC spectrometer measuring from 600 to 1000 nm in the double beam mode with untreated gasoline as the reference. The absorption maximum was 738 nm and the absorbance was 218. Serial dilutions of the base solution gave linear decreases of absorbance in accordance with Beers Law. The minimum detectable concentration was near 19 ppb.

Molecular Marker

The molecular marker should have good thermal stability and little light absorption in the visible region; that is they should impart little or no color to the liquid to which the molecular marker is copolymerized or admixed with. Also, they should have strong absorption of near infrared light (high molar extinction coefficients, e.g., >20,000) and have strong fluorescence in the near infrared over the wavelengths of about 670-2500 nm. To produce essentially “invisible” tags the near infrared fluorescent compounds must absorb little if any light having wavelengths in the 400-670 nm range; however, since the compounds are present in extremely low concentrations, a small amount of absorption may be tolerated without imparting significant color.

Preferred molecular markers can be organic compounds, preferably those which are compatible in small amounts with the intended use of the liquid and are soluble in the liquid in at least small amounts. For petroleum products, preferred compounds may contain O, N, S or P in addition to C and H, since such elements are typically found in petroleum products. Preferably, all elements contained in the molecular markers are found at natural isotopic abundance, since the elements are found in the petroleum products at natural isotopic abundance. The molecular markers are employed in sufficiently small amounts that their presence is not detectable in the bulk material without the use of sensitive analytic instruments and not easily detectable even with analytic instruments unless their identity is known. Suitable materials are generally oleophilic and hydrophobic, since the petroleum product compositions are generally oleophilic and hydrophobic.

Molecular markers suitable for use can generally be described as non-radioactive compounds. Preferably, the molecular markers used can be detected in the liquid which contains them at concentrations of less than 500 ppb, such as in the 0.5-500 ppb range.

It is desirable to assemble a library or collection of suitable molecular markers and make selections from the library to formulate markers based on compatibility of the molecular markers with the liquid to be marked and the use of a molecular marker.

Two classes of suitable materials which can be used as molecular markers are polynuclear aromatic hydrocarbons and halogenated hydrocarbons. Examples of suitable marking agents include 1,2-diphenylbenzene; 1,4-diphenylbenzene, triphenylmethane, 1,3,5-triphenylbenzene, 1,1,2-triphenylethylene; tetraphenylethylene, 1,2,3,4-tetrahydrocarbazole, 1,3-diphcnylacetone, 2-chlorobenzophenone; 4,4′-dichlorobenzophenone; 4-benzoylphenone; 4-bromobenzophenone; 4-methoxybenzophenone; 4-methylbenzophenone; 9-fluorenone; 1-phenylnaphthalene; 3,3′dimethoxybiphenyl; and 9-phenylanthracene.

Compatibility is rather easy to determine and is based on the range of properties of the liquid to be transported or stored. It does not require an especially large collection of marking agents to accomplish the capability to provide unique combinations. For example, 1,000 tagging agents can be used to formulate over 41 billion unique 3-component markers.

The following example illustrates further the synthetic methods which are used in preparing the compounds which are useful in the practice of the invention.

### EXAMPLE 4

3.2 Kg of o-terphenyl was added to a 14,000 gallon container of gasoline additive. A previous chemical analysis of the gasoline intended to be tagged had revealed the absence of o-terphenyl at detection limits. The additive was metered into the gasoline product at the ratio of ½ gallon of additive per 1,000 gallons of gasoline. The resulting concentration of the chemical tag in the gasoline product into which the additive has been metered was calculated to be 40 ppb. This was confirmed by chemical analysis.

Other molecular markers can include molecules where the molecular weight of the molecule is artificially enchanced. For example, any element or compound which can be produced with stable isotopes not generally found in nature is suitable for the molecular marker. The molecular marker is labeled with a non-radioactive atom at least one specific site in the molecule. Particularly preferred are those compounds deuterated or rendered isotopic by carbon-13 or fluorine-19. Also preferred are nitrogen-15, oxygen-17 and oxygen-18 isotopic materials.

The molecular marker is more commonly a non-radioactive isotope of such organic solvents as acetone, acetonitrile, benzene, bromobenzene, chlorobenzene, chloroform, cyclohexane, dichlorobenzene, trichloroethylene, diethylether, diglyme, dimethylsulfoxide, dioxane, ethanol, methanol, methylene chloride, nitrobenzene, octane, pyridine, tetrachloroethane, tetrahydrofuran, tetrametholsilane, toluene, trifluoroacetic acid, trifluoroethyl alcohol, xylene, ammonium bromide, or acetyl chloride.

One particularly preferred class of organic compounds are those which have been deuterated, i.e., wherein the hydrogen atoms covalently bound to carbon atoms are replaced with deuterium atoms. Deuterium is a non-radioactive isotope of hydrogen which is often called heavy hydrogen. Deuteration of organic compounds can be accomplished by methods known in the art such as those disclosed in U.S. Pat Nos. 3,746,634 and 3,876,521 wherein deuteration is effected with deuterium gas in the presence of a Group VII or VIII metal catalyst at a temperature between about 100 and about 300 degrees C. The non-radioactive isotopes for use in this invention may further be prepared in accordance with the prior art teachings of such materials used in the medical arts. The non-radioactive chemical substance may have the heavy atom in any position of the molecule. Likewise, one or more of the reactive sites of a molecule may contain a heavy atom. For example, the number of permutations possible with n-octane is in the thousands since one or all of the hydrogen atoms of the molecule may be substituted with deuterium as set forth below:
CH₂DCH₂CH₂CH₂CH₂CH₂CH₂CH₃;
CH₃CHDCH₂CH₂CH₂CH₂CH₂CH₃;
CH₂DCHDCH₂CH₂CH₂CH₂CH₂CH₃;
CH₂DCH₂CH₂CH₂CH₂CH₂CH₂CH₂D;
CH₂DCHDCHDCH₂CH₂CH₂CH₂CH₃;
CH₂DCHDCHDCH₂CH₂CH₂CH₂CH₂D.
The number of uniquely identifiable combinations of deuterated n-octanes naturally decreases the chance that more than one liquid will contain the same non-radioactive isotope.

### EXAMPLE 5

A 100,000 dead weight ton tanker is filled with crude oil for shipment to its desired location. About one eighth of one quart of deuterated octane of the formula CH₂DCH₂CH₂CH₂CH₂CH₂CH₂CH₃ is added to the crude oil. This provides approximately one part per billion of deuterated octane in the tanker. Upon arrival at its destination point, a sample of crude oil is removed from the tanker for testing.

Once the markers are dissolved and in a liquid form they are combined into a single-combination marker. The combination marker is either directly added to the petroleum product or diluted by the petroleum product and then added to the petroleum product. For example, to add the marker from Example 3 and the marker from Example 4 to 14,000 gallons of gasoline, 24.4 kg grams of dimethy lindole cyanine and 3.2 kg of o-terphenyl was added directly to the gasoline resulting in a solution of roughly 305 ppb dimethy lindole cyanine and 40 ppb o-terphenyl. If the combination marker is added to a transportation container, the transporting of the fuel will properly mix the combination marker with the petroleum product. For example, gasoline is typically in a tanker truck and the natural transport will properly mix the added marker. However, if the petroleum product is in a stationary tank, then some sort of physical mixing such as Vaughan's Rotamix system or some other mixing process known in the art may have to be performed into order to achieve a uniform mixture of the petroleum product and the added marker.

For example and not by way of limitation, if the petroleum product is in a pipe line, the combination marker may be injected into the petroleum stream through an injection valve as the petroleum product flows from the processing plant to a storage container. If the petroleum product is in a tanker truck or ship, the combination marker may be added by opening a port to the transport container and adding the marker directly through the port. Also, the combination marker could be injected into the transport container below the fuel level. The petroleum product may also be in a static storage container either above ground or below ground. If the petroleum product is in static storage container, the combination marker may be added by opening a port to the static container and adding the marker directly through the port or the marker could be injected into the container below the fuel level. The combination marker may also be added to a dry container before the petroleum product is added. In the case of diesel fuel, it may be preferable to add the combination marker at the same time a coloring dye is added at the taxation terminal.

FIG. 1 shows the preferred embodiment of the method to test a marked liquid for adulteration. At Step 100, a set of samples is collected from a group of liquids to be tested. For example, samples of gasoline from different gasoline stations. At Step 101, a target absorbance level is selected for instance, 0.5 of course, depending on the absorbance marker chosen this target may vary. Second, a tolerance is selected, Step 102. For example, if ±10% tolerance for the absorbance level of the absorbance marker is acceptable, then the tolerance would be 90%-110%. If the absorbance of the absorbance marker is below 90% or above 110% then the concentration of the absorbance marker would be outside the range of acceptability. Next, a test is preformed to determine the absorbance of the liquid in the 600-1000 wavelength range to determine the concentration of the absorbance marker, Step 104. The absorption is directly related to concentration of the absorbance marker in the liquid. The test for the presence of the added absorbance marker includes obtaining a sample of the gasoline and testing it in a portable IR spectrometer. The presence and concentration of the marker is indicated by a peak on the IR spectrum at the known wavelength for the marker. Portable IR spectrometers are commercially available from Isotag Technology, Inc. of Dallas, Tex. At Step 105, a decision is made as to whether or not the absorbance is within the pre-selected tolerance. If the absorption peak of the sample is inside the pre-selected tolerance a random test of the samples is done for the molecular marker, Step 106. Of course a systematic testing of the samples could be done as well. If the absorption peak of the sample is outside the pre-selected tolerance, then each sample is tested for the molecular marker, Step 108. The results of the chromatographic analysis are compared with the results from the absorption field test, Step 110. If the results agree, then the absorption test results are accepted, and the field equipment is validated, Step 112. If the results of the chromatographic analysis and the absorption field test do not agree, then the chromatographic results are accepted as valid and the absorption field test equipment results are rejected, Step 114. Calibration of the field equipment may be checked in this manner.

The method of the invention results in an efficient method to screen samples qualitatively through field equipment to arrive at a percentage of samples which do not require testing in a laboratory in order to arrive at a reliable indication of the identification and quality of the liquid in all the samples.

FIG. 2 provides an example of an application of the method described in FIG. 1.

FIG. 2 shows a spectrograph of two different samples, 202 and 204, of a marked kerosene. Plot 202 indicates, at 206 an absorbtivity of 0.5 at a wavelength of 780 nm. If, for example, the chosen target was 0.5 at 780 nm and the chosen tolerance was ±10%, then curve 202 would be inside the tolerance range and the sample which produced plot 202 would “pass” as an unadulterated liquid. Alternatively, the plot in 204 shows an absorbance of approximately 1.6 at a wavelength of 780 nm. This absorbance would be outside the tolerance range and therefore the liquid which produced curve 204 would be considered a “fail”.

Although the invention has been described with reference to one or more preferred embodiments, this description is not to be construed in a limiting sense. There is modification of the disclosed embodiments, as well as alternative embodiments of this invention, which will be apparent to persons of ordinary skill in the art, and the invention shall be viewed as limited only by reference to the following claims.

1-5. (cancelled)

6. A method for marking a liquid comprising the steps of:
adding a marker to the liquid having a molar absorptivity of approximately 5 times 10⁴ L mole⁻¹ cm⁻¹ or greater in the wavelength range of about 600-1000 nm.

7. The method of claim 6 wherein the liquid is a petroleum product.

8. The method of claim 6 wherein the molar absorptivity is in the range of approximately 5×10⁴ L moles⁻¹ cm⁻¹ to 10⁵ L mole⁻¹ cm⁻¹.

9. The method of claim 6 wherein the desired concentration of the marker is between 1 ppb and 10 ppm.

10. The method of claim 6 wherein the marker produces a characteristic peak at a known wavelength.

11. The method of claim 6 wherein the marker contains a compound selected from the group consisting essentially of metal containing and metal free phthalocyanine, metal containing and metal free naphthalocyanine, squarilium, croconic acid, indole and substituted indole cyanine and carbocyanine, thiazole type cyanine and carbocyanine, oxazole type cyanine and carbocyanine, metal dithiolene complexes, and indoaniline metal complexes.

12. A method for marking a liquid comprising the steps of:
adding a first marker to the liquid having a molar absorptivity of approximately 5 times 10⁴ L mole⁻¹ cm⁻¹ or greater in the wavelength range of about 600-1000 nm; and
adding to the liquid a second marker wherein the second marker is a molecular marker.

13. The method of claim 12 wherein the liquid is a petroleum product.

14. The method of claim 12 wherein the desired concentration of the first marker is between 1 ppb and 10 ppb.

15. The method of claim 12 wherein the first marker contains a compound selected from the group consisting essentially of metal containing and metal free phthalocyanine, metal containing and metal free naphthalocyanine, squarilium, croconic acid, indole and substituted indole cyanine and carbocyanine, thiazole type cyanine and carbocyanine, oxazole type cyanine and carbocyanine, metal dithiolene complexes, and indoaniline metal complexes.

16. The method of claim 12 wherein the second marker is non-radioactive.

17. The method of claim 12 wherein the second marker is a polynuclear aromatic hydrocarbon.

18. The method of claim 12 wherein the second marker is a halogenated hydrocarbon.

19. The method of claim 12 wherein the second marker is selected from the group consisting essentially of 1,2-diphenylbenzene, 1,4-diphenylbenzene, triphenylmethane, 1,3,5-triphenylbenzene, 1,1,2-triphenylethylene, tetraphenylethylene, 1,2,3,4-tetrahydrocarbazole, 1,3-diphcnylacetone, 2-chlorobenzophenone, 4,4′-dichlorobenzophenone; 4-benzoylphenone; 4-bromobenzophenone, 4-methoxybenzophenone, 4-methylbenzophenone, 9-fluorenone, 1-phenylnaphthalene, 3,3′dimethoxybiphenyl, and 9-phenylanthracene.

20. The method of claim 12 wherein the molecular weight of the second marker is artificially enhanced.

21. The method of claim 12 wherein the molecular weight is enhanced by the addition of a deuterium atom.

22. The method of claim 12 wherein the second marker is selected from the group consisting essentially of acetone, acetonitrile, benzene, bromobenzene, chlorobenzene, chloroform, cyclohexane, dichlorobenzene, trichloroethylene, diethylether, diglyme, dimethylsulfoxide, dioxane, ethanol, methanol, methylene chloride, nitrobenzene, octane, pyridine, tetrachloroethane, tetrahydrofuran, tetrametholsilane, toluene, trifluoroacetic acid, trifluoroethyl alcohol, xylene, ammonium bromide, and acetyl chloride.

23. A method for marking a liquid comprising the steps of:
adding a first marker to the liquid having a molar absorptivity of approximately 5 times 10⁴ L mole⁻¹ cm⁻¹ or greater in the wavelength range of about 600-1000 wherein the first marker contains a compound selected from the group consisting essentially of metal containing and metal free phthalocyanine, metal containing and metal free naphthalocyanine, squarilium, croconic acid, indole and substituted indole cyanine and carbocyanine, thiazole type cyanine and carbocyanine, oxazole type cyanine and carbocyanine, metal dithiolene complexes, and indoaniline metal complexes; and
adding a second marker to the liquid wherein the second marker is selected from the group consisting essentially of 1,2-diphenylbenzene, 1,4-diphenylbenzene, triphenylmethane, 1,3,5-triphenylbenzene, 1,1,2-triphenylethylene, tetraphenylethylene, 1,2,3,4-tetrahydrocarbazole, 1,3-diphcnylacetone, 2-chlorobenzophenone, 4,4′-dichlorobenzophenone; 4-benzoylphenone; 4-bromobenzophenone, 4-methoxybenzophenone, 4-methylbenzophenone, 9-fluorenone, 1-phenylnaphthalene, 3,3′dimethoxybiphenyl, and 9-phenylanthracene.

24. The method of claim 23 wherein the liquid is a petroleum product.

25. The method of claim 23 wherein the desired concentration of the first marker is between 1 ppb and 10 ppm.

26. A method for marking a liquid comprising the steps of:
adding a first marker to the liquid having a molar absorptivity of approximately 5 times 10⁴ L mole⁻¹ cm⁻¹ or greater in the wavelength range of about 600-1000 wherein the first marker contains a compound selected from the group consisting essentially of metal containing and metal free phthalocyanine, metal containing and metal free naphthalocyanine, squarilium, croconic acid, indole and substituted indole cyanine and carbocyanine, thiazole type cyanine and carbocyanine, oxazole type cyanine and carbocyanine, metal dithiolene complexes, and indoaniline metal complexes; and
adding a second marker to the liquid wherein the molecular weight of the second marker is artificially enhanced by the addition of a deutirium atom from the group consisting essentially of acetone, acetonitrile, benzene, bromobenzene, chlorobenzene, chloroform, cyclohexane, dichlorobenzene, trichloroethylene, diethylether, diglyme, dimethylsulfoxide, dioxane, ethanol, methanol, methylene chloride, nitrobenzene, octane, pyridine, tetrachloroethane, tetrahydrofuran, tetrametholsilane, toluene, trifluoroacetic acid, trifluoroethyl alcohol, xylene, ammonium bromide, and acetyl chloride.

27. The method of claim 26 wherein the liquid is a petroleum product.

28. The method of claim 26 wherein the desired concentration of the first marker is between about 1 ppb and 10 ppm.

29. A method for determining if samples of a liquid have been adulterated comprising the steps of:
adding a first marker to the samples having a molar absorptivity in the wavelength range of about 600-1000 nm in a first predetermined concentration;
adding a second marker to the samples in a second predetermined concentration wherein the second marker is a molecular marker;
testing the samples to determine the concentration of the first marker;
determining if the concentration of the first marker is within a pre-selected tolerance; and
if the concentration of the first marker is not within the pre-selected tolerance, testing the samples to determine the concentration of the second marker to determine if the samples have been adulterated.

30. The method of claim 29 wherein the liquid is a petroleum product.

31. The method of claim 29 wherein the first marker has a molar absorptivity of greater than approximately 5 times 10⁴ L mole⁻¹ cm⁻¹.

32. The method of claim 29 wherein the testing of the concentration of the first marker includes using an IR spectrometer.

33. The method of claim 32 wherein the IR spectrometer is a portable IR spectrometer.

34. The method of claim 29 wherein the concentration of the first marker is indicated by a peak in the IR spectrum of the tested sample.

35. The method of claim 29 wherein the first marker contains a compound selected from the group consisting essentially of metal containing and metal free phthalocyanine, metal containing and metal free naphthalocyanine, squarilium, croconic acid, indole and substituted indole cyanine and carbocyanine, thiazole type cyanine and carbocyanine, oxazole type cyanine and carbocyanine, metal dithiolene complexes, and indoaniline metal complexes.

36. The method of claim 29 wherein the first predetermined concentration is between 1 ppb and 10 ppb.

37. The method of claim 29 wherein the second marker is non-radioactive.

38. The method of claim 29 wherein the second marker is a polynuclear aromatic hydrocarbon.

39. The method of claim 29 wherein the second marker is a halogenated hydrocarbon.

40. The method of claim 29 wherein the second marker is an organic compound containing elements found in natural isotopic abundance.

41. The method of claim 29 wherein the second maker is a chemical compound which is nonexistent in nature and can be identified in liquids using gas chromatograph/mass spectrometer techniques at concentrations in the range of 1 to 100 parts per billion.

42. The method of claim 29 wherein the second marker has been labeled with a non-radioactive atom located at one specific site in a molecule of the molecular marker.

43. The method of claim 29 wherein the second marker is selected from the group consisting essentially of 1,2-diphenylbenzene, 1,4-diphenylbenzene, triphenylmethane, 1,3,5-triphenylbenzene, 1,1,2-triphenylethylene, tetraphenylethylene, 1,2,3,4-tetrahydrocarbazole, 1,3-diphcnylacetone, 2-chlorobenzophenone, 4,4′-dichlorobenzophenone, 4-benzoylphenone, 4-bromobenzophenone, 4-methoxybenzophenone, 4-methylbenzophenone, 9-fluorenone, 1-phenylnaphthalene, 3,3′-dimethoxybiphenyl, and 9-phenylanthracene.

44. The method of claim 29 wherein the molecular weight of the second marker is artificially enhanced.

45. The method of claim 44 wherein the molecular weight is enhanced by the addition of a deuterium atom.

46. The method of claim 29 wherein the second marker is selected from the group consisting essentially of acetone, acetonitrile, benzene, bromobenzene, chlorobenzene, chloroform, cyclohexane, dichlorobenzene, trichloroethylene, diethylether, diglyme, dimethylsulfoxide, dioxane, ethanol, methanol, methylene chloride, nitrobenzene, octane, pyridine, tetrachloroethane, tetrahydrofuran, tetrametholsilane, toluene, trifluoroacetic acid, trifluoroethyl alcohol, xylene, ammonium bromide, and acetyl chloride.

47. The method of claim 29 wherein the second predetermined concentration is less than 500 ppb.

48. The method of claim 29 wherein the pre-selected tolerance is about 90-110% of the predetermined concentration of the first marker.

49. The method of claim 29 further comprising the step of randomly choosing a subset of samples if the concentration of the first marker is within the pre-selected tolerance and testing the subset of samples to determine the concentration of the second marker to determine if the samples have been adulterated.

50. The method of claim 29 wherein the concentration of the second marker is determined from a chromatographic/mass spectrometer analysis.

51. A liquid marker compound comprising:
a first marker having a molar absorptivity of approximately 5×10⁴ L moles⁻¹ cm⁻¹ or greater in the wavelength range of about 600 to 1000 nm; and
a second marker wherein the second marker is a molecular marker.

52. A liquid marker of claim 51 wherein a desired concentration of the first marker is between 1 ppb and 10 ppm.

53. The liquid marker of claim 51 wherein the first marker produces a characteristic peak at a known wavelength.

54. The liquid marker of claim 51 wherein the first marker contains a compound selected from the group consisting essentially of metal containing and metal free phthalocyanine, metal containing and metal free naphthalocyanine, squarilium, croconic acid, indole and substituted indole cyanine and carbocyanine, thiazole type cyanine and carbocyanine, oxazole type cyanine and carbocyanine, metal dithiolene complexes, and indoaniline metal complexes.

55. A liquid marker of claim 51 wherein the second marker is a polynuclear aeromatic hydrocarbon.

56. The liquid marker of claim 51 wherein the second marker is a halogenated hydrocarbon.

57. The liquid marker of claim 51 wherein the second marker is selected from the group consisting essentially of 1,2-diphenylbenzene, 1,4-diphenylbenzene, triphenylmethane, 1,3,5-triphenylbenzene, 1,1,2-triphenylethylene, tetraphenylethylene, 1,2,3,4-tetrahydrocarbazole, 1,3-diphcnylacetone, 2-chlorobenzophenone, 4,4′-dichlorobenzophenone; 4-benzoylphenone; 4-bromobenzophenone, 4-methoxybenzophenone, 4-methylbenzophenone, 9-fluorenone, 1-phenylnaphthalene, 3,3′dimethoxybiphenyl, and 9-phenylanthracene.

58. The liquid marker of claim 51 wherein the molecular weight of the second marker is artificially enhanced.

59. The liquid marker of claim 58 wherein the molecular weight is enhanced by the addition of a deuterium atom.

60. The liquid marker of claim 51 wherein the second marker is selected from the group consisting essentially of acetone, acetonitrile, benzene, bromobenzene, chlorobenzene, chloroform, cyclohexane, dichlorobenzene, trichloroethylene, diethylether, diglyme, dimethylsulfoxide, dioxane, ethanol, methanol, methylene chloride, nitrobenzene, octane, pyridine, tetrachloroethane, tetrahydrofuran, tetrametholsilane, toluene, trifluoroacetic acid, trifluoroethyl alcohol, xylene, ammonium bromide, and acetyl chloride.

61. The liquid marker of claim 51 wherein the presence of the first molecular marker can be determined by a handheld IR spectrometer.

62. A method of testing authenticity of plurality of samples comprising the steps of:
performing a first test of the plurality for a first marker;
comparing the results of the first test to a predetermined standard;
if the results of the first test do not meet the predetermined standard, performing a second test on the plurality for a second marker to determine authenticity;
if the results of the first test meet the predetermined standard, randomly selecting a sample from the plurality and performing the second test on the sample for the second marker to determine authenticity.

63. The method of claim 62 wherein the first test is an optical test.

64. The method of claim 62 wherein the first test is a test for absorbance.

65. The method of claim 62 wherein the second test is a chemical test.

66. The method of claim 62 wherein the second test is a chromatographic test.

67. The method of claim 62 comprising the further steps of:
comparing the results of the first test with the result of the second test;
adopting the results of the second test if the results of the first test and the results of the second test do not agree.

68. the method of claim 67 comprising the further step of:
validating a test device if the results of the first test and the results of the second test agree.
